(19) United States
(12) Patent Application Publication
(10) Pub. No.: US 2012/0146252 A1
(43) Pub. Date: Jun. 14, 2012

(54) CONTROL METHOD FOR DETERMINING CURE STATUS OF GLASS FIBER PRODUCTS

(75) Inventors: Christopher M. Hawkins, Alexandria, OH (US); Donald R. Miller, Granville, OH (US); Ronald Cseh, New Albany, OH (US); David Shallenberger, Newark, OH (US)

(73) Assignee: Owens Corning Intellectual Capital, LLC, Toledo, OH (US)

(21) Appl. No.: 13/313,071

(22) Filed: Dec. 7, 2011

Related U.S. Application Data
(60) Provisional application No. 61/421,295, filed on Dec. 9, 2010.

(51) Int. Cl. B29C 47/92; G01N 33/00

(52) U.S. Cl. 264/40.1; 73/159

(57) ABSTRACT

A method for assessing the cure status of a fibrous blanket manufactured with mineral fibers and binder is disclosed and comprises a first, qualitative assessment of cure followed by a second quantitative assessment that utilizes one of alternative procedures depending on the outcome of the first, qualitative assessment. For example, a first visual inspection may qualitatively reveal areas or undercure, upon which a second pH test of samples extracted from undercured areas may quantify the degree of undercure. Conversely, if the visual inspection reveals no undercured areas, a different sampling procedure examines different blanket areas for overcure status using a similar pH test.

This application claims priority to provisional application 61/421,295, filed Dec. 9, 2010.

### BACKGROUND

This invention relates in general to insulation products made from fibrous minerals like glass and, in particular, to quality control methods for determining the cure status, i.e. whether the product is undercured, overcured or properly cured within specifications and process control limits.

Fibrous glass insulation products generally comprise randomly-oriented glass fibers bonded together by a cured thermosetting polymeric material. Molten streams of glass are drawn into fibers of random lengths and blown into a forming chamber or hood where they are randomly deposited as a pack onto a porous, moving conveyor or chain. The fibers, while in transit in the forming chamber and while still hot from the drawing operation, are sprayed with an aqueous dispersion or solution of binder. The residual heat from the glass fibers and from the flow of air during the forming operation are sufficient to vaporize much of the water from the binder, thereby concentrating the binder dispersion and depositing binder on the fibers as a viscous liquid with high solids content. Ventilating blowers create negative pressure below the conveyor and draw air, as well as any particulate matter not bound in the pack, through the conveyor and eventually exhaust it to the atmosphere. The uncured fibrous pack is transferred to a curing oven where a gas, heated air for example, is blown through the pack to cure the binder and rigidly bond the glass fibers together in a random, three-dimensional structure, usually referred to as a “blanket.” Sufficient binder is applied and cured so that the fibrous pack can be compressed for packaging, storage and shipping, yet regains its thickness—a process known as “loft recovery”—when compression is removed.

While manufacturers strive for rigid process controls, the degree of binder cure throughout the pack may not always be uniform for a variety of reasons. Irregularities in the moisture of the uncured pack, irregularities in the flow or convection of drying gasses in the curing oven, uneven thermal conductance from adjacent equipment like the conveyor, and non-uniform applications of binder, among other reasons, may all contribute to areas of over- or under-cured binder. Thus it is desirable to test for these areas in final product to assure quality.

U.S. Pat. No. 7,063,983 teaches a method of assessing the cure status of polycarboxylic acid binders using a pH indicator solution (nitrazine) that turns yellow or purple, depending if the pH is below or above, respectively, a value between 6.5 and 6.8. While this binary method gives a simple qualitative measure of degree of cure, it does not give complete quantitative information about the cure and gives no real information about over-cured status. Moreover, it does not help the manufacturer know whether to scrap the product or merely to adjust the process controls to bring the process back within the process control limits.

### SUMMARY OF THE INVENTION

This invention relates generally to methods for assessing the cure status of a fibrous blanket manufactured with mineral fibers and binder. In one aspect, the invention comprises a method of determining the cure status of a mineral fiber product comprising:

making a first qualitative assessment of cure status to identify a representative sample;

making a second quantitative assessment of cure status, wherein the sampling procedure for making the second quantitative assessment depends on the result of the first qualitative assessment.

The first qualitative assessment may be a visual inspection for a representative sample, such as an undercured area, which may appear as unusual, lighter or darker color, or dense spots in the mineral fiber product. Alternatively, the first qualitative assessment may be an indicator solution that exhibits a first color indicating an undercured state and a second color indicating a cured state.

The second quantitative assessment uses information or results from the first qualitative assessment to test the representative sample. In one variation, the first qualitative assessment may inform how to take the second sample or from where to take it. For example, if an undercured area is perceived, the second test may include extracting a sample from an area that appears to be undercured. Conversely, if the first qualitative assessment indicates a cured state, the procedure for the second quantitative assessment includes extracting a sample from an area that may potentially be overcured, such as an edge or outer layer. In another variation, the first qualitative assessment may inform which test to apply as the second quantitative assessment. While many quantitative assessments are possible, one convenient one is based on absolute pH measurement.

The result of the second quantitative assessment is generally used to inform at least one decision regarding the mineral fiber product, such as to accept or reject the tested batch or to make one or more process adjustments for manufacturing subsequent mineral fiber product. Thus, in a second aspect, the invention comprises a method of monitoring and adjusting the manufacturing process controls in a process for making mineral fiber products, said method comprising:

attenuating molten mineral into fibers and collecting the fibers in a pack of randomly oriented mineral fibers, applying a binder, and curing the pack to form a blanket, all under process controls having predetermined process control limits;

making a first qualitative assessment for possible undercured areas of the blanket;

making a second quantitative assessment of cure status of the blanket, the procedure for which depends on the result of the first qualitative assessment; and

adjusting at least one process control in response to the result of the second quantitative assessment of cure status.

As with the first aspect, the first qualitative assessment may be a visual inspection for a representative sample, such as an undercured area, which may appear as unusual, lighter or darker color, or dense spots in the mineral fiber product. Alternatively, the first qualitative assessment may be an indicator solution that exhibits a first color indicating an undercured state and a second color indicating a cured state. Similarly, the procedure for the second quantitative assessment uses information or results from the first qualitative assessment in order to test the representative sample. The results of the first assessment may dictate the location of procedure for taking a test sample and/or the nature of the second quantitative assessment. In the case of undercure results or findings, this may include extracting a sample from an area that appears to be undercured. Conversely, if the first qualitative assessment indicates a cured state, the procedure for the second quantitative assessment includes extracting a sample from an area that may potentially be overcured, such as an edge or outer layer. While many quantitative assessments are possible, one convenient one is based on absolute pH measurement.

Process control decisions that may be made in response to the result of the second quantitative assessment of cure status potentially include adjusting the process control to bring the process back within the predetermined process control limits, and this may be accomplished in either the oven or the forming hood area. For example, a process adjustment might mean adjusting in at least one zone of a curing oven an oven parameter selected from temperature, air flow, and residence time in the oven zone. Alternatively, a process adjustment might mean adjusting at least one forming area parameter selected from coolant flow, binder flow, air flow, and residence time in the forming area.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a partially sectioned side elevation view of a forming hood component of a manufacturing line for manufacturing fibrous products;

FIG. 2 is a flow diagram representing the steps of one process embodiment according to the invention; and

FIG. 3A-3B is a flow diagram representing the steps of a second process embodiment according to the invention.

### DETAILED DESCRIPTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. All references cited herein, including books, journal articles, published U.S. or foreign patent applications, issued U.S. or foreign patents, and any other references, are each incorporated by reference in their entireties, including all data, tables, figures, and text presented in the cited references.

In the drawings, the thickness of the lines, layers, and regions may be exaggerated for clarity.

Unless otherwise indicated, all numbers expressing ranges of magnitudes, such as angular degrees or sheet speeds, quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements. All numerical ranges are understood to include all possible incremental sub-ranges within the outer boundaries of the range. Thus, a range of 30 to 90 degrees discloses, for example, 35 to 50 degrees, 45 to 85 degrees, and 40 to 80 degrees, etc.

“Binders” are well known in the industry to refer to thermosetting organic agents or chemicals, often polymeric resins, used to adhere glass fibers to one another in a three-dimensional structure that is compressible and yet regains its loft when compression is removed. “Binder delivery” refers to the mass or quantity of “binder chemical” e.g. “binder solids” delivered to the glass fibers. This is typically measured in the industry by loss on ignition or “LOL” which is a measure of the organic material that will burn off the fibrous mineral. A fibrous pack is weighed, then subjected to extreme heat to burn off the organic binder chemical, and then reweighed. The weight difference divided by the initial weight (×100) is the % LOI.

As solids, rate of binder delivery is properly considered in mass/time units, e.g. grams/minute. However, binder is typically delivered as an aqueous dispersion of the binder chemical, which may or may not be soluble in water. “Binder dispersions” thus refer to mixtures of binder chemicals in a medium or vehicle and, as a practical matter, delivery of binder “dispersions” is given in flow rate of volume/time. e.g. liters/minute or LPM of the dispersion. The two delivery expressions are correlated by the mass of binder per unit volume, i.e. the concentration of the binder dispersion. Thus, a binder dispersion having X grams of binder chemical per liter flowing at a delivery rate of Z liters per min delivers X*Z grams/minute of binder chemical. Dispersions include true solutions, as well as colloids, emulsions or suspensions.

One specific type of binder dispersion—referred to as a “binder concentrate”—is a stock dispersion having a relatively high, fixed concentration, e.g. 20-40%, of binder solids that can be subsequently diluted with a binder “diluent” (typically more water) to produce a diluted “binder dispersion” having a lower concentration, e.g. 10%, of binder. This diluted, “ultimate” binder dispersion is then sprayed or dispensed on the glass fibers. A constant delivery of binder chemical (grams/minute) may still be achieved by a higher flow rate of a more dilute binder dispersion. The term “binder dispersion” is generic for both the ultimate, diluted form and the concentrated stock form. Binder dispersions of 25-30% solids may be used for some commercial products, while binder dispersions of 5-15% solids may be used for other products, such as residential products. Binder tackiness and viscosity in the forming hood are important properties impacting product properties, and are dependent on the concentration (% solids), the particular binder chemistry and the temperature.

References to “acidic binder” or “low pH binder” mean a binder having a dissociation constant (Ka) such that in an aqueous dispersion the pH is less than 7, generally less than about 6, and more typically less than about 4.

“Mineral fibers” refers to any mineral material that can be melted to form molten mineral that can be drawn or attenuated into fibers. Glass is the most commonly used mineral fiber for fibrous insulation purposes and the ensuing description will refer primarily to glass fibers, but other useful mineral fibers include rock, slag and basalt.

“Product properties” refers to a battery of testable physical properties that insulation batts possess. These may include at least the following common properties:
“Recovery”—which is the ability of the batt or blanket to resume it's original or designed thickness following release from compression during packaging or storage.

It may be tested by measuring the post-compression height of a product of known or intended nominal thickness, or by other suitable means.
“Stiffness” or “sag”—which refers to the ability of a batt or blanket to remain rigid and hold its linear shape. It is measured by draping a fixed length section over a fulcrum and measuring the angular extent of bending deflection, or sag. Lower values indicate a stiffer and more desirable product property. Other means may be used.
“Tensile Strength”—which refers to the force that is required to tear the fibrous product in two. It is typically measured in both the machine direction (MD) and in the cross machine direction (“CD” or “XMD”).
“Lateral weight distribution” (LWD or “cross weight”)—which is the relative uniformity or homogeneity of the product throughout its width. It may also be thought of as the uniformity of density of the product, and may be measured by sectioning the product longitudinally into bands of equal width (and size) and weighing the band, by a nuclear density gauge, or by other suitable means.
“Vertical weight distribution” (VWD)—which is the relative uniformity or homogeneity of the product throughout its thickness. It may also be thought of as the uniformity of density of the product, and may be measured by sectioning the product horizontally into layers of equal thickness (and size) and weighing the layers, by a nuclear density gauge, or by other suitable means.
   Of course, other product properties may also be used in the evaluation of final product, but the above product properties are ones found important to consumers of insulation products.

The nouns “assessment”, “evaluation” and “test”, as well as verb and adjective forms thereof, may be used interchangeably when referring to a process for estimating or determining the cure status of a pack or blanket.

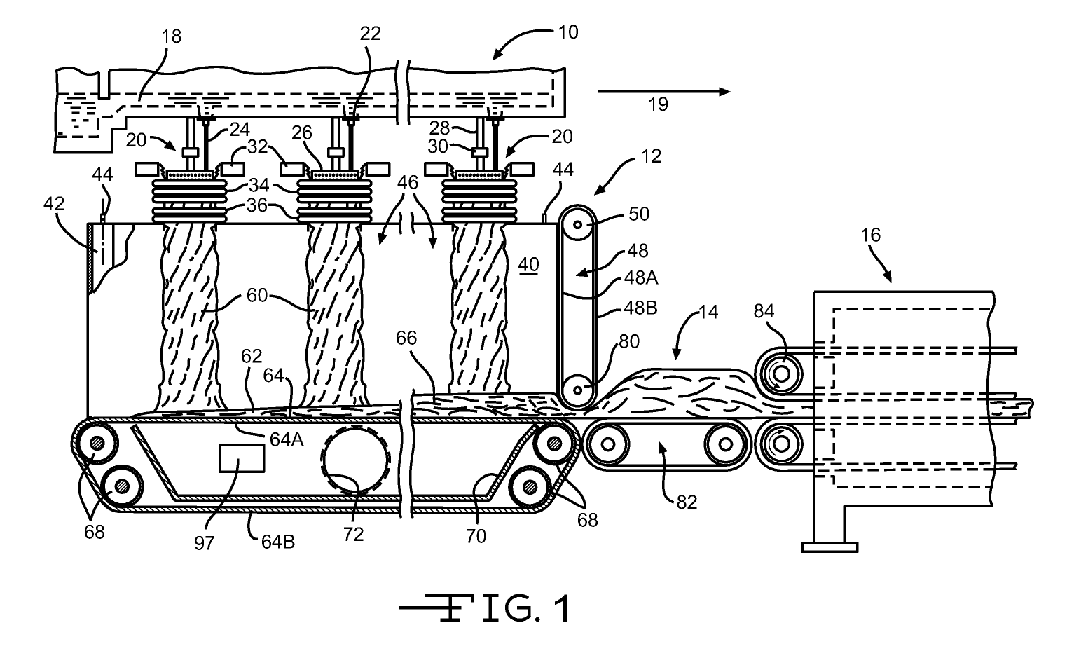

FIG. 1 illustrates a glass fiber insulation product manufacturing line including a forehearth 10, forming hood component or section 12, a ramp conveyor section 14 and a curing oven 16. Molten glass from a furnace (not shown) is led through a flow path or channel 18 to a plurality of fiberizing stations or units 20 that are arranged serially in a machine direction, as indicated by arrow 19 in FIG. 1. At each fiberizing station, holes 22 in the flow channel 18 allow a stream of molten glass 24 to flow into a spinner 26, which may optionally be heated by a burner (not shown). Fiberizing spinners 26 are rotated about a shaft 28 by motor 30 at high speeds such that the molten glass is forced to pass through tiny holes in the circumferential sidewall of the spinners 26 to form primary fibers. Blowers 32 direct a gas stream, typically air, in a substantially downward direction to impinge the fibers, turning them downward and attenuating them into secondary fibers that form a veil 60 that is forced downwardly. The fibers are distributed in a cross-machine direction by mechanical or pneumatic “lappers” (not shown), eventually forming a fibrous layer 62 on a porous conveyor 64. The layer 62 gains mass (and typically thickness) with the deposition of additional fiber from the serial fiberizing units, thus becoming a fibrous “pack” 66 as it travels in a machine direction 19 through the forming area 46.

One or more cooling rings 34 spray coolant liquid, such as water, on veil 60 to cool the fibers within the veil. Other coolant sprayer configurations are possible, of course, but rings have the advantage of delivering coolant liquid to fibers throughout the veil 60 from a multitude of directions and angles. A binder dispensing system includes binder sprayers 36 to spray binder onto the fibers of the veil 60. Illustrative coolant spray rings and binder spray rings are disclosed in US Patent Publication 2008-0156041 A1, to Cooper. Each fiberizing unit 20 thus comprises a spinner 26, a blower 32, one or more cooling liquid sprayers 34, and one or more binder sprayers 36. FIG. 1 depicts three such fiberizing units 20, but any number may be used. For insulation products, typically from two to about 15 units may be used in one forming hood component for one line.

The forming area 46 is further defined by side walls 40 and end walls 48 (one shown) to enclosed a forming hood. The side walls 40 and end walls 48 are each conveniently formed by a continuous belt that rotates about rollers 44 or 50, 80 respectively. The terms “forming hoodwall”, “hoodwall” and “hood wall” may be used interchangeably herein. Inevitably, binder and fibers accumulate in localized clumps on the hoodwalls and, occasionally, these clumps may fall into the pack and cause anomalous dense areas or “wet spots” that are difficult to cure.

The conveyor chain 64 contains numerous small openings allowing the air flow to pass through while links support the growing fibrous pack. A suction box 70 connected via duct 72 to fans or blowers (not shown) are additional production components located below the conveyor chain 64 to create a negative pressure and remove air injected into the forming area. As the conveyor chain 64 rotates around its rollers 68, the uncured pack 66 exits the forming section 12 under exit roller 80, where the absence of downwardly directed airflow and negative pressure (optionally aided by a pack lift fan, not shown) allows the pack to regain its natural, uncompressed height or thickness s. A subsequent supporting conveyor or “ramp” 82 leads the fibrous pack toward an oven 16 and between another set of porous compression conveyors 84 for shaping the pack to a desired thickness for curing in the oven 16. Upon exit from the oven 16, the cured pack or “blanket” (not shown) is conveyed downstream for cutting and packaging steps. For some products, the blanket is split longitudinally into multiple lanes and then chopped into shorter segments known as “batts.” These may be bundled or rolled for packaging.

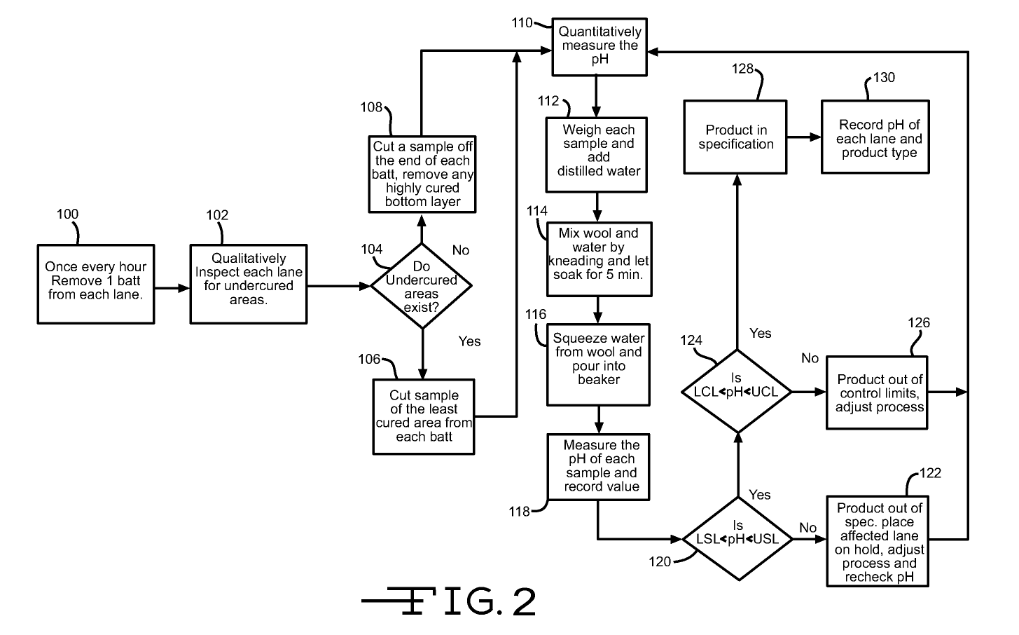

In accordance with the present invention the cured blanket or batt is sampled to determine the degree of cure in a more quantitative way. Referring to FIG. 2, a first, embodiment of the inventive process is described. At a predetermined frequency, the blanket or batt is sampled, as indicated at 100. The process is two-staged in that a first, qualitative assessment is made, followed by a second, quantitative determination. The first stage ensures that a representative sample is selected; and the second stage builds on this result to provide a quantitative measure that provides guidance on direction and magnitude of a process change should the result not be within specifications and/or process control limits.

The first, qualitative assessment may be a visual inspection to look for a suitable, representative sample, as noted at 102. Visual inspections may include assessments based on color, texture or consistency of the blanket. For products from Owens Corning, a dye is typically added to the binder and undercured areas will appear as a lighter shade of pink. For other manufacturers, the colors and shades may vary, or the visual inspection may rely on compressed or denser areas or other irregularities or spots that indicate an undercured state. Those working in this art are quite skilled at spotting undercured areas, if they exist.

As an alternative to mere visual inspection, an indicator solution may be used. As noted in U.S. Pat. No. 7,063,983 to Chen, et al., a dilute nitrazine solution may be used as an indicator solution to estimate pH qualitatively. A nitrazine indicator solution turns yellow or purple, depending if the pH is below or above, respectively, a value between about 6.5 and about 6.8. While this binary method gives a simple qualitative measure of degree of cure, it does not give complete quantitative information about the cure and gives no real information about over-cured status. While a visual inspection is simplest, other qualitative assessments may also be employed that lead one skilled in the art to select a representative sample from the cured fibrous pack or “blanket.”

Depending on the results of the qualitative assessment, at least one quantitative assessment is performed next; and the details of how the second, quantitative assessment is performed will be guided by the outcome of the first, qualitative test. This guidance may come in at least two variations. In one embodiment, information or results from the first assessment guide how the sample is taken or from what part of the blanket it is taken. In a second embodiment, information or results from the first assessment guide which second test to perform as the second assessment.

In the first embodiment, if the first, qualitative assessment indicates an undercured state, a sampling procedure is used that attempts to quantify the degree of undercure. Such a sampling procedure will select for further testing one or more areas of the blanket that appear in the first assessment to be undercured. Conversely, if the qualitative assessment indicates no undercured state, a sampling procedure is used that attempts to quantify the degree, if any, of overcured state. In this case, the sampling procedure examines the ends, edges, top or bottom layers or other exposed areas that might tend to be overcured. These sample areas are then subjected to a second, quantitative assessment of the cure state. The desire to evaluate for overcure is particularly important in the case of natural binders made from starches, dextrins, maltodextrins, carbohydrates and the like, because overcure of these binders may cause undesirable product properties such as discoloration or malodorous products. Such natural binders are disclosed in commonly owned U.S. patent application Ser. No. 12/900,540, filed Oct. 8, 2010, published Apr. 14, 2011 as US patent publication 2011/0086567, and incorporated by reference.

In a variation, if the first, qualitative assessment indicates an undercured state, this information may guide the nature of the second, quantitative test. For example, it may encompass the pH test mentioned here, or a visual or optical test; whereas a first indication of overcure might trigger a second assessment based on pH, odor, optical, chromatography or other analytical technique. Although many such second, quantitative tests are possible, one of the simplest is a pH test and the example of a quantitative pH test will be used in the following description. Other quantitative tests may include acid titration, colorimetric analysis, moisture analysis, and thermal history, either continuously made while the line is running or intermittently made by selecting periodic or random samples. As noted, the second, quantitative test may be the same or different depending on the outcome of the qualitative test and on the sample suggested by that first test.

Referring still to the embodiment of FIG. 2, the method of sampling varies, depending on the result of the first, qualitative assessment, but the method of qualitative assessment in this embodiment does not vary. Step 104 queries the result of the qualitative test: was an undercured area found or suspected? If yes, then step 106 instructs how to prepare a sample for the second, quantitative test. More specifically, a predetermined size sample—for example 8×8×2 inches—is taken from the area that appears to be the least cured area. This sample size may vary, of course, but should be large enough to produce a representative sample and small enough that reagents are not unduly consumed in the quality control testing. Furthermore, a single sample may be prepared by blending portions taken from different lanes and/or different areas of the blanket, such as edge or interior, top or bottom, etc. In some cases, multiple lane top sections may be blended as one sample, and multiple lane bottom sections may be blended as one sample.

If no undercured area is found upon qualitative inspection, the sample is prepared differently, as shown at step 108. In this case, the second stage test involves consideration of an overcure state rather than undercure, and certain areas (edge faces, top and bottom layers, etc) are more likely to be overcured than other areas. For the second stage test then, a sample is selected from one of the potentially overcured areas, such as an edge face (longitudinal or transverse faces) or top or bottom layer, or an end portion. A sample of predetermined dimension is removed from each batt and, to avoid skewing the result, any highly cured bottom layer is removed prior to testing. The bottom layer is sometimes more cured due to a variety of possible reasons, including, e.g. upward convection of high temperature air in the initial zone of the oven and conduction of additional heat from the conveyor chain 64 as the pack traverses the oven.

The sample then progresses to a quantitative, second stage evaluation which, as noted above, may conveniently be an absolute pH test. The specific procedure used for absolute pH determination is not critical, but a calibrated pH probe is one potential methodology. In one embodiment the batt sample is weighed (112) and a quantity of distilled water is added. Sufficient distilled water should be used to dissolve any uncured binder from the fibrous pack; for example, step 112 suggests ten times the weight of the batt. The water and fibrous pack should be mixed for a sufficient period of time to allow uncured binder to dislodge from the glass fibers and dissolve. At step 114, the fibrous pack or “wool” is kneaded and allowed to soak for at least 5 minutes. After the predetermined sufficient time has elapsed, the wool is removed and squeezed to extract the water into a suitable container, step 116. Thereafter, at step 118 the pH of the resulting extract solution is measured quantitatively to produce an absolute pH value. As noted above, a pH probe is one potential way to measure pH quantitatively.

With an absolute pH value in hand, the cure status of the pack or batt is known with a higher degree of accuracy, including information about the degree or magnitude of undercure or overcure, if any. This provides the manufacturer with valuable and actionable data with which to adjust the process controls as needed. For example, manufacturers have predetermined product specifications and product not falling within those ranges is said to be “out of spec” and must generally be scrapped. This is also referred to herein as a “reject” situation. Moreover, most manufacturers have process controls and set predetermined limits to the variability of their processes. These parameters, along with illustrative values, are summarized in the following Table 1.

TABLE 1
Manufacturing Limits

| | | Illustrative | One |
|---|---|---|---|
| Abbreviation | Term and meaning | pH level | Embodiment |
| USL | Upper Specification Limit - the | 6.8-7.2 | 6.9 |
| | value above which product is | | |
| | out of spec and must be | | |
| | discarded or scrapped. | | |
| UCL | Upper Control Limit - the value | 6.2-6.8 | 6.5 |
| | above which product is outside | | |
| | of the preset limits of acceptable | | |
| | process variability, although it | | |
| | may still be within spec. | | |
| LCL | Lower Control Limit - the value | 5.4-5.8 | 5.6 |
| | below which product is outside | | |
| | of the preset limits of acceptable | | |
| | process variability, although it | | |
| | may still be within spec. | | |
| LSL | Lower Specification Limit - the | 5.2-5.4 | 5.3 |
| | value below which product is | | |
| | out of spec and must be | | |
| | discarded or scrapped. | | |

Knowing the cure status quantitatively in relation to these limits has significant consequences for the manufacturer. As noted above, product that is “out of spec” is generally scrapped. But if the only information available to the manufacturer is that the pH is “low”—i.e. the product is undercured—then a manufacturer may scrap product unnecessarily if it was low but still above a LSL. More specifically, product testing outside the USL and LSL still must be scrapped, but product testing between the USL and UCL, or between the LCL and LSL may still be used and not scrapped. This is valuable information, since the manufacturer will incorrectly scrap good product less frequently.

Perhaps even more importantly, the manufacturer now gains quantitative information about how far the product is from any of the limits mentioned above. Previously, if product was within specification it was retained and the process was deemed acceptable and not necessarily adjusted. Product testing outside the Control Limits (i.e. >UCL or <LCL) but still within spec (i.e. >LSL and <USL) gives the manufacturer the opportunity to adjust process controls to try to bring the process back under tighter control. This is also referred to as a “react” situation, and knowing the test result quantitatively provides information about how much to adjust the process controls in the react circumstance. In other words, the quantitative result provides information not only about the direction of a process change, but also about the magnitude of such a process change. None of this is possible with simple, qualitative testing procedures.

Referring again to FIG. 2, the use of such quantitative information to make manufacturing process decisions is illustrated beginning with step 120. Step 120 asks if the test sample pH is “within spec,” i.e. is it true that LSL<pH<USL. If no, then the product is “out of spec” and must be discarded. Additionally, as shown at step 122, the production line producing “out of spec” product is halted until the problem can be remedied. However, if the response from step 120 is yes, then a further question is asked at step 124: is the test sample pH within the process control limits, i.e. is it true that LCL<pH<UCL? If no, then the product is within spec and need not be discarded, but the process is “out of control” and process adjustments should be made to bring the process back into control, i.e. back into conditions that produce acceptable variability within the process control limits, as noted at step 126. If the response at step 124 is yes, then the product is within spec and within process control limits (step 128), which is the desired state. After recording the details about the product and its pH (step 130) this product may proceed to packaging and sale. The cycle repeats according to a predetermined sampling frequency (step 100).

Potential adjustments that might be made to the process in response to the second quantitative assessment (e.g. pH) are highly variable, and include adjustments to the curing ovens as well as adjustments to the forming process itself. Curing oven adjustments may include the temperature set points, the air flow rate, and the residence time in the oven. Curing ovens are frequently divided into zones and such adjustments may be at one, some or each of the oven zones. Adjustments that might be made in the forming hood include, for example, using more or less coolant liquid, more or less binder liquid, altering the pH of any of the above solutions, altering the speed of the forming conveyor to change the residence time in the forming hood, and altering the rate of air flow caused by the blowers and the negative pressure suction boxes.

### EXAMPLE 1

This procedure is used to evaluate an insulation product for cure. Product will be placed on hold/scrapped if product pH is less than LSL (5.25) or greater than USL (6.9). The product is judged for undercure first using the Low Cure Qualitative Evaluation procedure below (step 1). If undercured areas are found, the Low Cure Quantitative Evaluation procedure is performed (step 2); but if there are no undercured areas the tester continues with a High Cure Evaluation as described in the next section (step 3). The tester should not perform both quantitative evaluations but only the applicable test. The test should be performed once every hour by collecting batts from all lanes simultaneously.

1. Low Cure Qualitative Evaluation: (a) tester visually inspects the batts from all lanes for uncured (dark pink) or undercured areas (the lightest pink). (b) tester may optionally test these for cure by spraying the suspect area of the least cured batt with pH indicator solution. In either case, the tester should examine the edges of all lanes for the worst spot apart from obvious dropped hoodwall clumps or “wet spots”.

2. Low Cure Quantitative Evaluation: (a) If there are no undercured areas larger than the equivalent of a 3″ diameter circle (˜9 sq in.), indicated visually or by the indicator solution turning from blue to yellow, this step is skipped and step 3 is performed instead. However, if such undercured areas are indicated visually or by the indicator solution turning from blue to yellow, a pH test should be run on the least cured spot for each lane. This involves tearing the batt in half looking for the least cured spot and cutting an 8″ square from the batt. (b) The pH probe is calibrated immediately prior to each use with standard buffer solutions prepared freshly each 24 hours. Standard buffers of at least two pH values are prepared: pH 7 and pH 4, for example. The probe is inserted into the first buffer solution, which is stirred or swirled for at least 20 seconds prior to pressing a button for machine calibration. The probe is rinsed with distilled water and blotted dry between each buffer solution. (c) The pH test should be conducted immediately following calibration and all tests should be completed as quickly as possible to avoid potential pH shift with time. Samples taken from the batt are weighed and should weigh at least 5 grams. The sample is kneaded well with ten times its weight (+/−10%) of distilled water, and after 5 minutes, the sample is squeezed to obtain a minimum of 30 g extract which is tested for pH within 5 minutes. The probe is rinsed with distilled water and blotted dry for the next test.

Results of the pH test are used as follows: The target pH is between 5.8 and 6.2 across all lanes. Steps should be taken to raise cure level of the affected lanes if uncured levels are detected. If any individual lane pH is less than LCL (5.5), the process should be adjusted to raise the level of cure. If the pH is less than LSL (5.25), the material made since the last good result must be isolated and scrapped. The pH of each lane should be rechecked once the adjustments are made to the process and it is stable.

3. High Cure Quantitative Evaluation: If there are no low cure areas, as detected visually or by the spraying of the indicator solution in Step 1, the operator should proceed as follows (and not test the lowest cured spot). (a) Cut off a 2″ piece from the end of each batt from each lane and, if necessary, remove no more than ½″ off the bottom surface to eliminate any overcure influence. (b) Run a pH on this sample using the standard pH testing procedure as outlined in steps 2(b) and 1(c) above.

React to the pH test results as follows: The target pH is between 5.8 and 6.2 across all lanes. If the pH of any single lane is greater than USL (6.9), material made since the last good result must be isolated and scrapped. If any individual lane pH is greater than UCL (6.6), the process should be adjusted to reduce the level of cure. The operator should make process adjustments based on pH results but should also consider EOL stiffness and recovery results as well as lateral weight distribution (or “cross weights”) as inputs to what to do. To address non-uniform lateral weight distribution, the forming area lappers may require adjustment. After adjustments the pH should be rechecked once the oven settles out.

Note: the first step to reduce overall cure is to reduce oven parameters, like air flow or temperature, provided that end of line (“EOL”) product properties allows this. However, if decreasing the oven parameters does not achieve desirable cure status, then coolant or other liquid adjustments in the forming hood may be required instead.

### EXAMPLE 2-5

### Lane

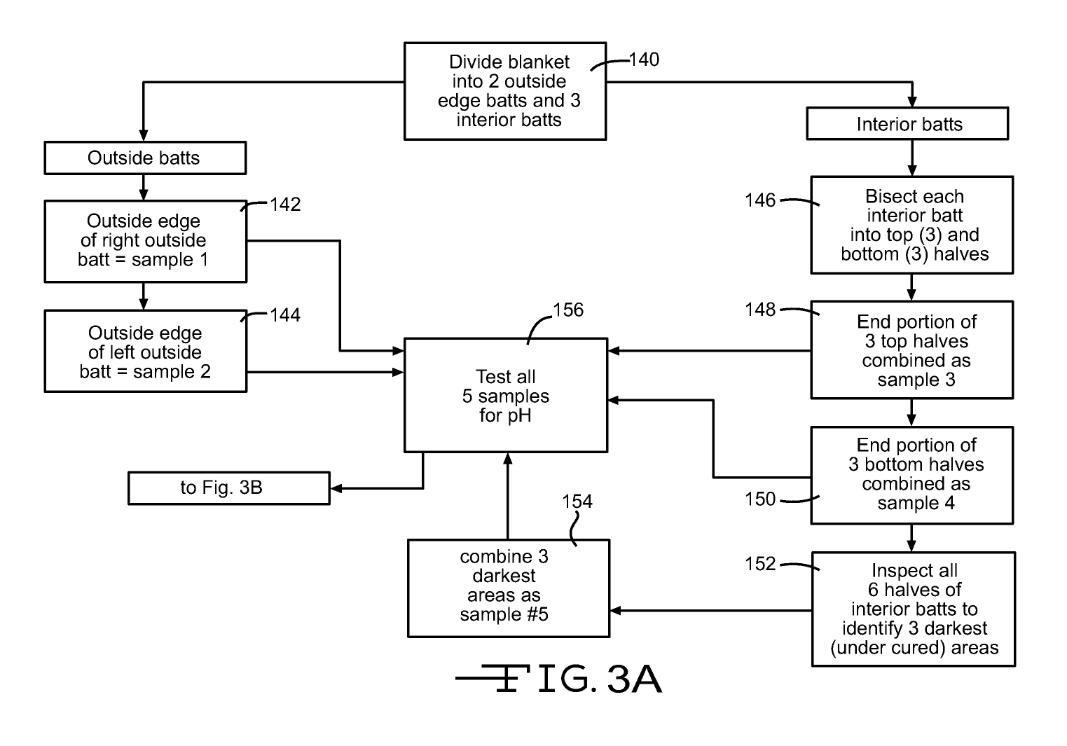

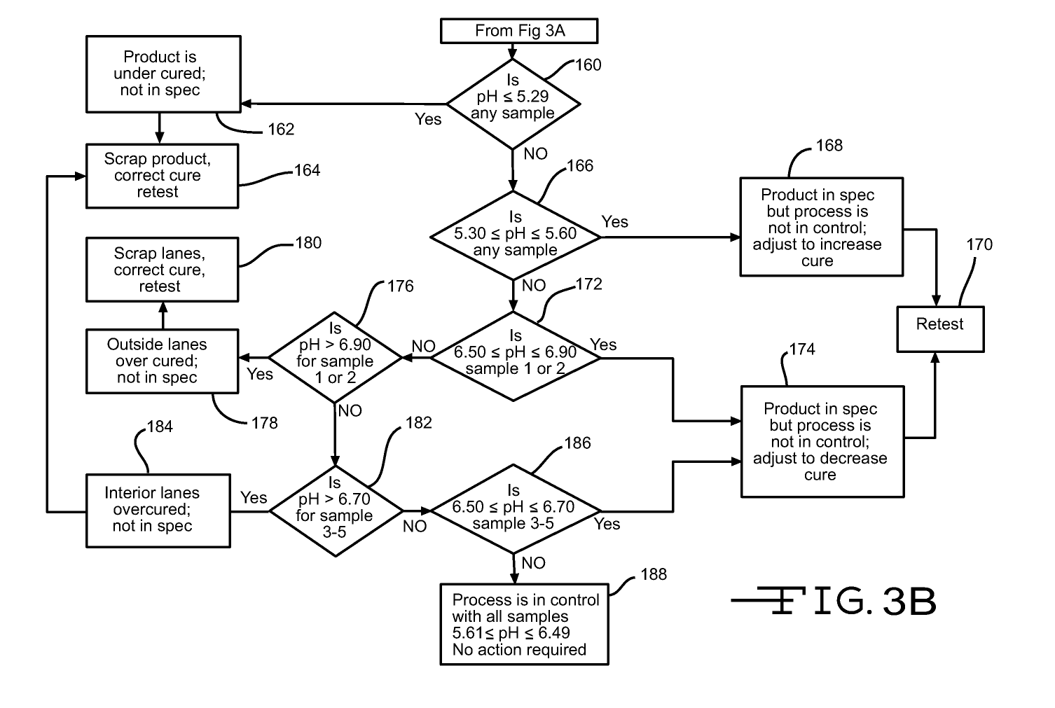

Another embodiment is depicted in FIG. 3A-3B. This embodiment is useful for a 5-lane manufacturing line, where the pack formed in the forming hood and cured in the oven is several feet wide and sufficient to support five “lanes” of batts as described above. Knives slice the blanket longitudinally into the five lanes and batts from the two outside lanes (designated “right” and “left” for convenience) are separated from bats from the three interior lanes, step 140. Sections of the outermost edges of the outside batts are taken as the first two samples. Arbitrarily, sample 1 from the right batt (142) and sample 2 from the left batt (144). These samples may be any shape or size, but should include the outermost edge of the batt. A strip of about 12 inches long and 2 inches into the batt has been found suitable.

Next, in either order, the interior batts are bisected into top (T) and bottom (B) halves (146) or a portion of the end is cut from each of the interior batts (148, 150). The result is six half-height end portions. The three from top halves are combined to make sample 3 (148) and three from bottom halves are combined to make sample 4 (150). The end portions should include the full width of the batt and extend about 1-2 inches into the end.

The remaining six interior half-height batts (3 top and 3 bottom) are subjected to the first qualitative assessment to look for uncured or under cured areas (152). The three darkest areas of the six half-height batts are identified for subjecting to the second, quantitative assessment (152). A portion of the batts in the three identified areas is excised and combined for testing. Again, any size sample may be taken, but an 8×8 inch square section has been found suitable. These three square are combined as sample 5, step 154. All 5 samples are passed on to the second qualitative test (156), which is described further in FIG. 3B.

FIG. 3B is similar to FIG. 2 in providing a flow diagram or decision tree. A first step (160) asks if any of the test sample 1-5 has a pH less than or equal to 5.29 (the LSL). If so, the product is “out of spec” (undercured step 162) and must be scrapped; line corrections would be made and new product retested (164). If the pH is at least 5.30 but not more than 5.60 (LCL), step 166 indicates the product is “within spec” but not as cured as one would like, so line adjustments are made to increase cure level (168). Some specific line adjustments that might be used are set forth in Example 3. Product is retested (170).

When the product tests above the LSL and LCL, then one must still consider if it is over the UCL or USL. Note that in this embodiment, the UCL is 6.50, but the USL is different for edge samples 1 and 2 than for interior samples 3, 4 and 5. Step 172 asks if the edge samples 1 and 2 have a pH between 6.5 and 6.9. If yes, they are “within spec”, but not in the ideal target range so “out” of control limits, 174. Step 176 asks if the edge samples 1 and 2 have a pH>6.9 and thus exceed the USL. If so, the outside lanes at least are overcured (178) and must be scrapped. Corrective action and retesting is required (180). Next, Step 182 asks similar questions about the interior lane samples 3, 4 and 5. If any of these have a pH of 6.7 or higher, they are out of spec and scrapped (184, 164). If not, step 186 asks how they stand relative to control limits (UCL). If any of them are between pH 6.5 and 6.7, the product is within spec, but the process is not within control, and adjustments to decrease cure are implemented (174). If all test blocks (160, 166, 172, 176, 182 and 186) result in “No” answers, logically the product is within the target pH range of 5.61 to 6.49 and the process is “in control” and no corrective action is needed (188).

For the pH test of this example, the pH probe is calibrated every 24 hours as described in Example 1 and/or in compliance with documentation for the instrument. The pH test should be conducted immediately following calibration (within 3 minutes) and all tests should be completed as quickly as possible to avoid potential pH shift with time. Samples taken from the batt are weighed and should weigh at least 5 grams. The sample is kneaded well with ten times its weight (+/−10%) of distilled water, and after 5 minutes, the sample is squeezed to obtain an extract which is tested for pH within 3 minutes.

Note: the first step to reduce overall cure is to reduce oven parameters, like air flow or temperature, provided that end of line (“EOL”) product properties allows this. However, if decreasing the oven parameters does not achieve desirable cure status, then coolant or other liquid adjustments in the forming hood may be required instead.

### EXAMPLE 3

### Selected Corrective Actions

The following Tables set forth some corrective actions to take in given situations depending on the cure status of various samples.

Process Issue: Bright Pink Areas in Interior Batts (Under Cure)

Action

Ensure proper weight distribution across all lanes
Look for plugged areas on the Oven Flights
Look for sources of excess moisture on the Forming Chain
Look for sources of excess moisture from the fiberizing area
Ensure that Oven fan speeds are optimized: run each fan as fast as
possible without blowing craters in the surface (updraft zones) or
degrading machine thickness (downdraft zones).

Process Issue: Interior Top is Under Cured

Action

Check for plugged areas on top oven chain
Verify Ramp Height is at target
Increase temperature in last two oven zones by 5° each (react zone) or
10° each (reject zone)
Increase fan speeds in last two oven zones by 50 rpm each - ensure that
pack is still touching top oven chain at discharge end and surface quality
is not affected

Process Issue: Interior Bottom is Under Cured

Action

Look for sources of excess moisture from the fiberizing area; especially
on initial units that from the “bottom” of pack.
Look for sources of excess moisture on forming chain - i.e. under chain
sprays, leaking hoses, etc.
Look for overflowing catch pans or hoodwall troughs
Ensure proper operation of forming chain cleaner sprayer
Ensure proper operation of forming flight dryer
Check for plugged areas on bottom oven chain
Verify Ramp Height is at target
Increase temperature in first two oven zones by 5° each (react zone) or
10° each (reject zone)
Increase fan speeds in first two oven zones by 50 rpm each - ensure that
surface quality is not degraded (blowing holes in pack) and pack is still
touching top oven chain at discharge

Process Issue: Edge is Under Cured

Action

Ensure hoodwalls are rotating and squeegees are drying the belt
If edge sprays are being used, reduce flow or turn off
Check for plugged area on top and bottom oven chains, especially the
edges
Ensure that pack is centered on the oven chain. If not, air will bypass
the pack through the open chain, reducing cure on that edge of the
pack.
Verify Ramp Height is at target
Verify deckles are in correct position (if applicable)
Increase temperature in first two oven zones by 5° each (react zone) or
10° each (reject zone) Note that this will also increase cure throughout
the pack, so ensure that this move will not create an over-cured
condition elsewhere!

Process Issue: Interior Top is Over Cured

Action

Verify Ramp Height is at target
Decrease temperature in last two oven zones by 5° each (react zone) or
10° each (reject zone)

Process Issue: Interior Bottom is Over Cured

Action

Verify Ramp Height is at target
Decrease temperature in first two oven zones by 5° each (react zone) or
10° each (reject zone)

Process Issue: Edge is Over Cured

Action

Ensure that pack is centered on the oven chain
Verify Ramp Height is at target
Verify deckles are in correct position (if applicable)
Decrease temperature in first two oven zones by 5° each. Note that this
will also decrease cure results for the other areas of the pack, so ensure
that this move will not create an under-cured condition elsewhere!
Ensure proper edge trim width

Product Issue: All Regions Under Cured

Action

Verify Ramp Height is at target
Increase all Oven Zone temps by 5° each (react zone) 10° each (react
zone)
If oven changes do not result in increased cure, verify ramp moisture
is in acceptable range for the line. Extreme ambient conditions may
result in the inability to properly cure product, at which time it is
recommended to change jobs.

Product Issue: All Regions Over Cured

Action

Verify Ramp Height is at target
Increase all Oven Zone temps by 5° each (react zone) 10° each (react
zone)

The principle and mode of operation of this invention have been explained and illustrated in its preferred embodiment. However, it must be understood that this invention may be practiced otherwise than as specifically explained and illustrated without departing from its spirit or scope.

What is claimed is:

1. A method of determining the cure status of a mineral fiber product comprising:
making a first qualitative assessment of cure status to identify a representative sample;
making a second quantitative assessment of cure status, wherein the second quantitative assessment is performed using at least one step that depends on the result of the first qualitative assessment.

2. The method of claim 1 wherein the first qualitative assessment is a visual inspection for undercured areas.

3. The method of claim 2, wherein the first assessment is based on color or dense spots in the mineral fiber product.

4. The method of claim 1 wherein the first qualitative assessment is an indicator solution that exhibits a first color indicating an undercured state and a second color indicating a cured state.

5. The method of claim 1 wherein, if the result of the first qualitative assessment indicates an undercured state, the procedure for the second quantitative assessment includes extracting a sample from an area that appears to be undercured.

6. The method of claim 1 wherein if the result of the first qualitative assessment indicates a cured state, the procedure for the second quantitative assessment includes extracting a sample from an area that may potentially be overcured.

7. The method of claim 6 wherein the sample is extracted from an edge face or layer of the mineral fiber product.

8. The method of claim 1 wherein the second quantitative assessment is a based on a measurement of pH.

9. The method of claim 1 further comprising using the result of the second quantitative assessment to make a decision regarding acceptability of the mineral fiber product.

10. The method of claim 1 further comprising using the result of the second quantitative assessment to make a process adjustment decision for manufacturing subsequent mineral fiber product.

11. A method of monitoring and adjusting the manufacturing process controls in a process for making mineral fiber products, said method comprising:
attenuating molten mineral into fibers and collecting the fibers in a pack of randomly oriented mineral fibers, applying a binder, and curing the pack to form a blanket, all under process controls having predetermined process control limits;
making a first qualitative assessment for possible undercured areas of the blanket;
making a second quantitative assessment of cure status of the blanket, the procedure for which depends on the result of the first qualitative assessment; and
adjusting at least one process control in response to result of the second quantitative assessment of cure status.

12. The method of claim 11 wherein the first qualitative assessment is a visual inspection.

13. The method of claim 11 wherein if the result of the first qualitative assessment indicates an undercured state, the procedure for the second quantitative assessment includes extracting a sample from an area that appears to be undercured.

14. The method of claim 11 wherein if the result of the first qualitative assessment indicates a cured state, the procedure for the second quantitative assessment includes extracting a sample from an area that may potentially be overcured.

15. The method of claim 11 wherein the second quantitative assessment is a based on a measurement of pH.

16. The method of claim 11 wherein, if the result of the second quantitative assessment of cure status indicates that the process is not within the predetermined process control limits, the step of adjusting at least one process control includes adjusting the process control to bring the process back within the predetermined process control limits.

17. The method of claim 11 wherein the step of adjusting at least one process control in response to result of the second quantitative assessment of cure status includes adjusting in at least one oven zone a curing oven parameter selected from temperature, air flow, and residence time in the oven zone.

18. The method of claim 11 wherein the step of adjusting at least one process control in response to result of the second quantitative assessment of cure status includes adjusting at least one forming area parameter selected from coolant flow, binder flow, air flow, and residence time in the forming area.
